(19) United States
(12) Patent Application Publication
(10) Pub. No.: US 2004/0132764 A1
(43) Pub. Date: Jul. 8, 2004

(54) Antibiotics for the treatment of infections in acidic environments

(75) Inventor: Hans Locher, Binningen (CH)

Correspondence Address: PALMER & DODGE, LLP, PAULA CAMPBELL EVANS, 111 HUNTINGTON AVENUE, BOSTON, MA, 02199, US

(73) Assignee: Morphochem Aktiengesellschaft fuer Kombinatorische Chemie

(21) Appl. No.: 10/690,890

(22) Filed: Oct. 22, 2003

Related U.S. Application Data
(60) Provisional application No. 60/420,810, filed on Oct. 23, 2002.

(51) Int. Cl. A61K 31/4709; A61K 31/4745

(52) U.S. Cl. 514/300; 514/312

(57) ABSTRACT

The present invention relates to the use of compounds, in which the pharmacophores of quinolone and oxazolidinone are chemically linked together through a linker that is stable under physiological conditions, for the treatment of bacterial infections in acidic environments (pH<7.0). The activity of these compounds is strongly increased at even slightly acidic conditions what makes them especially interesting for the treatment of infections in abscesses or inflamed tissues.

### RELATED APPLICATIONS

This application claims priority under 35 U.S.C. § 119(e) to U.S. Provisional Application Serial No. 60/420,810 filed Oct. 23, 2002, the entirety of which is incorporated herein by reference.

The present invention describes the use of compounds in which the pharmacophores of quinolone and oxazolidinone are chemically linked together through a linker that is stable under physiological conditions for the treatment of bacterial infections in acidic environments. The activity of these compounds is strongly increased at even slightly acidic conditions in the range of pH 6.5 to 6.8.

The intensive use of antibiotics has exerted a selective evolutionary pressure on microorganisms to produce genetically based resistance mechanisms. Modern medicine and socio-economic behavior exacerbates the problem of resistance development by creating slow growth situations for pathogenic microbes, e.g. artificial joints-related infections, and by supporting long-term host reservoirs, e.g. in immuno-compromised patients.

In hospital settings, an increasing number of strains of Staphylococcus aureus, Streptococcus pneumoniae, Enterococcus sp., and Pseudomonas aeruginosa, major sources of infections, are becoming multi-drug resistant and therefore difficult if not impossible to treat:

S. aureus is often β-lactam and quinolone resistant. Now even vancomycin-resistance has been observed.

S. pneumoniae is becoming resistant to penicillin and even to new macrolides.

Enteroccocci are often quinolone and vancomycin resistant and β-lactams were never efficacious against these strains. The only alternative is to use oxazolidinones but these compounds are not bactericidal and the safety margin is rather low. Further, even with these drugs, resistance already appears in clinical practice.

In addition, microorganisms that are causing persistent infections are increasingly being recognized as causative agents or cofactors of severe chronic diseases like peptic ulcers or heart diseases.

In contrast to Linezolid and Ciprofloxacin, the activity of the compounds described herein is strongly dependent on the extracellular pH, i.e. the lower the pH, the better the activity. This special effect is explained by an intra-bacterial accumulation of the compound which is dependent on the difference between inside and outside pH. This property contributes to potent antibacterial activity and is of great interest for the treatment of infections involving (slightly) acidic environments as e.g. found in abscesses or inflammated tissues (Konig, C.; Simmen, H. P.; Blaser, J.; Eur. J. Microbiol. Infect. Dis. 1993, 12, 519) or in the strongly acidic environment of Helicobacter pylori-associated chronic gastritis.

The present invention provides the use of compounds of Formula (I) for the treatment of bacterial infections at a pH <7.4, preferred pH<7.0:

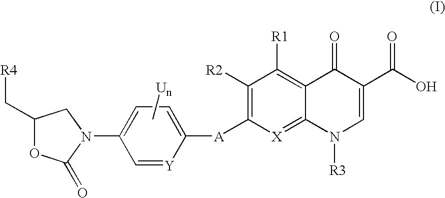

wherein

A is a direct bond, a NH, O, S, SO, SO₂, SO₂NH, PO₄, —NH—CO—NH—, —CO—NH—, —CO—, —CO—O—, —NH—CO—O—, an alkylene group, an alkenylene group, an alkinylene group, a heteroalkylene group, an arylene group, a heteroarylene group, a cycloalkylene group, a heterocycloalkylene group, an alkylarylene group or a heteroarylalkylene group or a combination of two or more of these atoms or groups;

X is CR5 or N;

Y is CR6 or N;

U is F or Cl;

n is 0, 1, 2 or 3;

R1 is H, F, Cl, Br, I, OH, NH₂, an alkyl group or a heteroalkyl group;

R2 is H, F or Cl;

R3 is a H atom or an alkyl group, an alkenyl group, an alkinyl group, a heteroalkyl group, a cycloalkyl group, a heterocycloalkyl group, an aryl group, a heteroaryl group, an alkylaryl group or a heteroarylalkyl group, all of which may be substituted with one, two or more halogen atoms like F or Cl.

R4 is a heteroalkyl group, a cycloalkyl group, a heterocycloalkyl group, an aryl group, a heteroaryl group, an alkylaryl group or a heteroarylalkyl group;

R5 is H, F, Cl, OH, NH₂, an alkyl group or a heteroalkyl group, or

R3 and R5 can be linked via an alkylene, an alkenylene or a heteroalkylene group or be a part of a cycloalkylene or heterocycloalkylene group, in case R3 is no H and R5 is no H, F, OH, NH₂ or Cl;

R6 is H, F, Cl or OMe;

or a pharmacologically acceptable salt, solvate, hydrate or formulation thereof.

It should be appreciated that certain compounds of formula (I) may have tautomeric forms from which only one might be specifically mentioned or depicted in the following description, different geometrical isomers (which are usually denoted as cis/trans isomers or more generally as (E) and (Z) isomers) or different optical isomers as a result of one or more chiral carbon atoms (which are usually nomenclatured under the Cahn-Ingold-Prelog or R/S system). Further, some compounds may display polymorphism. All these tautomeric forms, geometrical or optical isomers (as well as racemates and diastereomers) and polymorphous forms are included in the invention.

The term alkyl refers to a saturated straight or branched chain alkyl group, containing from one to ten, preferably one to six carbon atoms, for example methyl, ethyl, propyl, iso-propyl, butyl, iso-butyl, sec-butyl, tert-butyl, n-pentyl, iso-pentyl n-hexyl, 2,2-dimethylbutyl, n-octyl; ethenyl (vinyl), propenyl (allyl), iso-propenyl, n-pentyl, butenyl, isoprenyl or hexa-2-enyl; ethinyl, propinyl or butinyl groups. Any alkyl group as defined herein may be substituted with one, two or more substituents, for example F, Cl, Br, I, NH₂, OH, SH or NO₂.

The terms alkenyl and alkinyl refer to an unsaturated straight or branched chain alkyl group (having one, two or more double and/or triple bonds, an alkenyl preferably having one or two double bonds and an alkinyl preferably having one or two triple bonds), containing from two to ten, preferably two to six carbon atoms for example: ethenyl (vinyl), propenyl (allyl), iso-propenyl, n-pentenyl, butenyl, isoprenyl or hexa-2-enyl; ethinyl, propinyl or butinyl groups. Any alkenyl or alkinyl group as defined herein may be substituted with one, two or more substituents, for example F, Cl, Br, I, NH₂, OH, SH or NO₂.

The term heteroalkyl refers to an alkyl, alkenyl or alkinyl group as defined herein where one or more carbon atoms are replaced by an oxygen, nitrogen, phosphorous or sulphur atom for example an alkoxy group such as methoxy, ethoxy, propoxy, iso-propoxy, butoxy or tert.-butoxy, an alkoxyalkyl group such as methoxymethyl, ethoxymethyl, 1-methoxyethyl, 1-ethoxyethyl, 2-methoxyethyl or 2-ethoxyethyl, an alkylamino group such as methylamino, ethylamino, propylamino, isopropylamino, dimethylamino or diethylamino, an alkylthio group such as methylthio, ethylthio or isopropylthio or a cyano group. It may also refer to one of the above groups containing a keto group. The term heteroalkyl furthermore refers to a group derived from a carboxylic acid or carboxylic acid amide such as acetyl, propionyl, acetyloxy, propionyloxy, acetylamino or propionylamino, a carboxyalkyl group such as carboxymethyl, carboxyethyl or carboxypropyl, a carboxyalkyl ester, an alkylthiocarboxyamino group, an alkoxyimino group, an alkylaminothiocarboxyamino group or an alkoxycarbonylamino group. Any heteroalkyl group as defined herein may be substituted with one, two or more substituents, for example F, Cl, Br, I, NH₂, OH, SH or NO₂.

The term cycloalkyl refers to a saturated or partially unsaturated (having one, two or more double and/or triple bonds), cyclic group with one, two or more rings, having three to 14 carbon ring-atoms, preferably from five or six to ten carbon ring-atoms, for example cyclopropyl, cyclobutyl, cyclopentyl, cyclohexyl, tetralin, cyclopentenyl or cyclohex-2-enyl groups. Any cycloalkyl group as defined herein may be substituted with one, two or more substituents, for example F, Cl, Br, I, OH, NH₂, SH, N₃, NO₂, alkyl groups such as methyl or ethyl, heteroalkyl groups such as methoxy, methylamino, dimethylamino or cyanide.

The term heterocycloalkyl refers to a cycloalkyl group as defined herein where one, two or more carbon ring-atoms are replaced by one, two or more oxygen, nitrogen, phosphorous or sulphur atoms or S(O)₁₋₂ groups, for example piperidino, morpholino or piperazino groups.

The term aryl refers to an aromatic cyclic group with one, two or more rings, having five to 14 carbon ring-atoms preferably from five or six to ten carbon ring-atoms, for example phenyl or naphthyl groups. Any aryl group as defined herein may be substituted with one, two or more substituents, for example F, Cl, Br, I, OH, NH₂, SH, N₃, NO₂, alkyl groups such as methyl or ethyl, heteroalkyl groups such as methoxy, methylamino, dimethylamino or cyanide.

The term heteroaryl refers to an aryl group as defined herein where one, two or more ring-carbon atoms are replaced by an oxygen, nitrogen, boron, phosphorous or sulphur atom, for example pyridyl, imidazolyl, pyrazolyl, quinolinyl, isoquinolinyl, pyrrolyl, oxazolyl, isoxazolyl, thiazolyl, isothiazolyl, 1,2,3-triazolyl, 1,2,4-triazolyl, oxadiazolyl, thiadiazolyl, indolyl, indazolyl, tetrazolyl, pyrazinyl, pyrimidinyl and pyridazinyl groups.

The terms arylalkyl, alkylaryl and heteroarylalkyl, heteroalkylaryl refer to groups that comprise both aryl or, respectively, heteroaryl as well as alkyl and/or heteroalkyl and/or cycloalkyl and/or heterocycloalkyl groups.

Preferred and/or advantageous embodiments of the invention are subject-matter of the subclaims.

Preferred are compounds of Formula (I), wherein R1 is H or NH₂ (especially H).

Further preferred are compounds of Formula (I), wherein R2 is H or F (especially F).

Moreover preferred are compounds of Formula (I), wherein R3 is an ethyl, a 2-propyl, a C3-C6 cycloalkyl, a phenyl or a pyridyl group. All these groups may be substituted by one, two or more fluorine atoms or amino groups.

Moreover preferred are compounds of Formula (I), wherein R3 is a cyclopropyl group.

Further preferred are compounds of Formula (I), wherein R3 and R5 together form a bridge of the formula —O—CH₂—N(Me)- or —O—CH₂—CH(Me)—. Herein, the preferred stereochemistry at the chiral center is the one giving the (S) configuration in the final compound.

Further preferred are compounds of Formula (I), wherein R4 is a group of the formula —NHCOCH═CHAryl, —OHeteroaryl (especially -oxa-3-oxazol), —NHSO₂Me, —NHCOOMe, —NHCS₂Me, —NHCSNH₂, —NHCSOMe or —NHCOMe.

Especially preferred are compounds of Formula (I), wherein R4 is an acetylamino group.

Further preferred are compounds of Formula (I), wherein the absolute configuration at C-5 of the oxazolidinone ring is (S) according to the Cahn-Ingold-Prelog nomenclature system.

Moreover preferred are compounds of Formula (I), wherein R5 is H, F, Cl or a methoxy group which may be substituted by one, two or three fluorine atoms or a CF₃ group.

Further preferred are compounds of formula (I), wherein X is N or CH.

Further preferred are compounds of Formula (I), wherein Y is N or CF (especially CF).

Further preferred are compounds of Formula (I), wherein n is 0.

Further preferred are compounds of Formula (I), wherein A is a bond.

Further preferred are compounds of Formular (I), wherein A is a group of the formula

—B₀₋₁[D-E₀₋₁]ₘ-G₀₋₁-K₀₋₁—

wherein

the group B is NH, O, S, SO, SO₂, SO₂NH, an alkylene, which may be substituted by one, two or more fluorine atoms or a heteroalkylene group, which may be substituted by one, two or more fluorine atoms and/or at the optionally present nitrogen atoms by an alkyl or an acyl group;

the groups D independently of each other are optionally anellated heterocycloalkylene groups with 1, 2, 3 or 4 nitrogen atoms, which heterocycloalkylene groups may each be substituted by one, two or more fluorine atoms and/or which each may be substituted at one, two, three or four nitrogen atoms by an alkyl or an acyl group;

the groups E independently of each other are NH, O, S, SO, SO₂, SO₂NH, an alkylene, which may be substituted by one, two or more fluorine atoms or a heteroalkylene group, which may be substituted by one, two or more fluorine atoms and/or at the optionally present nitrogen atoms by an alkyl or an acyl group;

the group G contains one or more optionally anellated heterocycloalkylene groups with 1, 2, 3 or 4 nitrogen atoms, which heterocycloalkylene groups may each be substituted by one, two or more fluorine atoms and/or which each may be substituted at one, two, three or four nitrogen atoms by an alkyl or an acyl group;

the group K is NH, O, S, SO, SO₂, SO₂NH, an alkylene, which may be substituted by one, two or more fluorine atoms or a heteroalkylene group, which may be substituted by one, two or more fluorine atoms and/or at the optionally present nitrogen atoms by an alkyl or an acyl group; and m=1, 2, 3 or 4.

Moreover preferred are compounds of Formula (I), wherein A is a cycloalkylene or a alkylcycloalkylene group that contains 2, 3 or 4 heteroatoms (preferred O, N and S) and may be substituted by one, two or more fluorine atoms and the nitrogen atoms may be substituted by an alkyl or an acyl group.

Further preferred are compounds of Formula (I), wherein A is selected from the following groups which may be further substituted by one, two or more fluorine atoms or by one, two, three or four, preferably by one alkyl group which may be substituted by one, two or more fluorine atoms, and wherein the amino groups may be substituted by an alkyl or an acyl group:

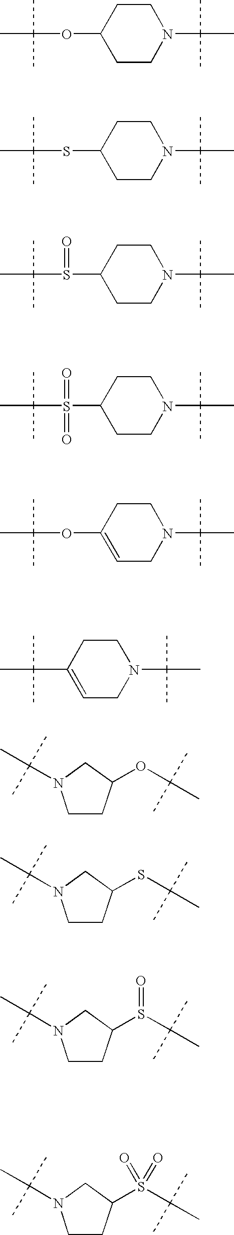

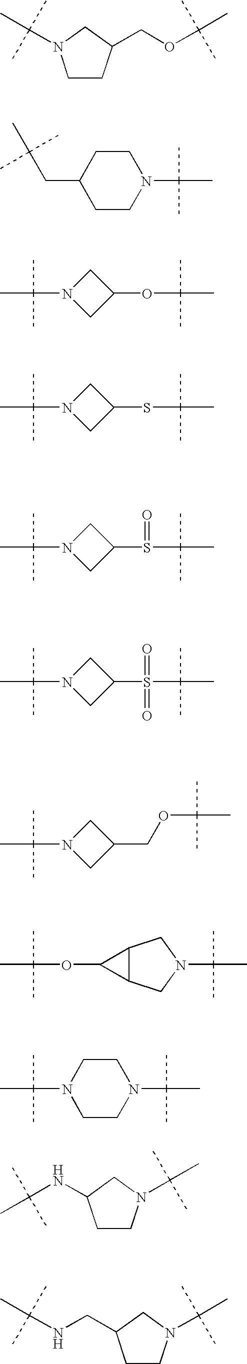

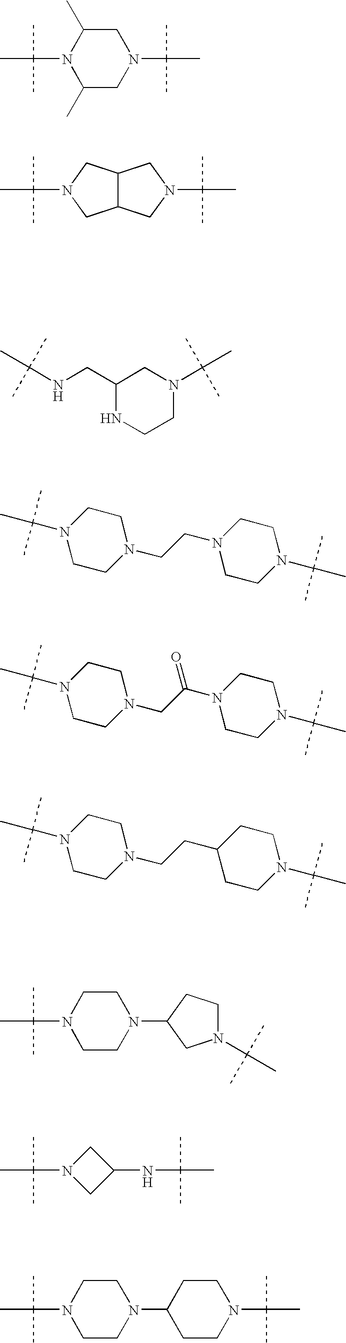

Moreover preferred are compounds of Formula (I), wherein A is a group of the formula —V—W—, wherein V is a direct bond or a group of the formula NH, O, S, SO, SO₂, SO₂NH, PO₄, —NH—CO—NH—, —CO—NH—, —CO—, —CH₂—, —CO—O—, —(CH₂)₁₋₃—O—, —CH═CH—C(O)—, or —NH—CO—O— and W is a heterocycloalkyl group with 4 to 7 ring atoms or an alkylheterocycloalkyl group with 4 to 7 ring atoms and 1 to 4 carbon atoms in the alkyl chain; all these groups may be substituted by 1, 2, 3 or 4 fluorine atoms, methyl or methoxy groups.

Further preferred are compounds of Formula (I), wherein A is a group of the formula

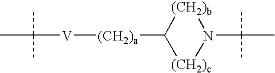

wherein V is a group of the formula NH, O, S, SO, SO₂, SO₂NH, PO₄, —NH—CO—NH—, —CO—NH—, —CO—, —CH₂—, —CO—O—, —(CH₂)₁₋₃—O—, —CH═CH—C(O)—, or —NH—CO—O—; a is 0, 1, 2, 3 or 4; b is 0, 1, 2, 3 or 4; c is 0, 1, 2, 3 or 4 and 1, 2, 3 or 4 hydrogen atoms may be substituted by F, a methyl- or a methoxy group.

Moreover preferred are compounds as described here, wherein V is NH, O, S, SO or SO₂.

Especially preferred are compounds as described here, wherein V is O or NH; a is 0 or 1; b is 1 or 2 and c is 1 or 2.

The present invention also relates to pharmacologically acceptable salts, or solvates and hydrates, respectively, and to compositions and formulations of compounds of Formula (I). The present invention describes procedures to produce pharmaceutically useful agents, which contain these compounds, as well as the use of these compounds for the production of pharmaceutically useful agents.

The pharmaceutical compositions according to the present invention contain at least one compound of Formula I as the active agent and optionally carriers and/or diluents and/or adjuvants. Optionally the pharmaceutical compositions according to the present invention may also contain additional known antibiotics.

Examples of pharmacologically acceptable salts of sufficiently basic compounds of Formula (I) are salts of physiologically acceptable mineral acids like hydrochloric, hydrobromic, sulfuric and phosphoric acid; or salts of organic acids like methanesulfonic, p-toluenesulfonic, lactic, acetic, trifluoroacetic, citric, succinic, fumaric, maleic and salicylic acid. Further, a sufficiently acidic compound of Formula (I) may form alkali or earth alkaline metal salts, for example sodium, potassium, lithium, calcium or magnesium salts; ammonium salts; or organic base salts, for example methylamine, dimethylamine, trimethylamine, triethylamine, ethylenediamine, ethanolamine, choline hydroxide, meglumin, piperidine, morpholine, tris-(2-hydroxyethyl)amine, lysine or arginine salts. Compounds of Formula (I) may be solvated, especially hydrated. The hydratisation can occur during the process of production or as a consequence of the hygroscopic nature of the initially water free compounds of Formula (I). The compounds of Formula (I) contain asymmetric C-atoms and may be present either as achiral compounds, mixtures of diastereomers, mixtures of enantiomers or as optically pure compounds.

The present invention also relates to pro-drugs which are composed of a compound of Formula (I) and at least one pharmacologically acceptable protective group which will be cleaved off under physiological conditions, such as an alkoxy-, aralkyloxy-, acyl-, acyloxymethyl group (e.g. pivaloyloxymethyl), an 2-alkyl-, 2-aryl- or 2-aralkyl-oxycarbonyl-2-alkylidene ethyl group or an acyloxy group as defined herein, e.g. ethoxy, benzyloxy, acetyl or acetyloxy.

As mentioned above, therapeutically useful agents that contain compounds of Formula (I), their solvates, salts or formulations are also comprised in the scope of the present invention. In general, compounds of Formula (I) will be administered by using the known and acceptable modes known in the art, either alone or in combination with any other therapeutic agent. Such therapeutically useful agents can be administered by one of the following routes: oral, e.g. as tablets, dragees, coated tablets, pills, semisolids, soft or hard capsules, for example soft and hard gelatine capsules, aqueous or oily solutions, emulsions, suspensions or syrups, parenteral including intravenous, intramuscular and subcutaneous injection, e.g. as an injectable solution or suspension, rectal as suppositories, by inhalation or insufflation, e.g. as a powder formulation, as microcrystals or as a spray (e.g. liquid aerosol), transdermal, for example via an transdermal delivery system (TDS) such as a plaster containg the active ingredient or intranasal. For the production of such tablets, pills, semisolids, coated tablets, dragees and hard, e.g. gelatine, capsules the therapeutically useful product may be mixed with pharmaceutically inert, inorganic or organic excipients as are e.g. lactose, sucrose, glucose, gelatin, malt, silica gel, starch or derivatives thereof, talc, stearinic acid or their salts, dried skim milk, and the like. For the production of soft capsules one may use excipients as are e.g. vegetable, petroleum, animal or synthetic oils, wax, fat, polyols. For the production of liquid solutions, emulsions or suspensions or syrups one may use as excipients e.g. water, alcohols, aqueous saline, aqueous dextrose, polyols, glycerin, lipids, phospholipids, cyclodextrins, vegetable, petroleum, animal or synthetic oils. Especially preferred are lipids and more preferred are phospholipids (preferred of natural origin; especially preferred with a particle size between 300 to 350 nm) preferred in phosphate buffered saline (pH=7 to 8, preferred 7.4). For suppositories one may use excipients as are e.g. vegetable, petroleum, animal or synthetic oils, wax, fat and polyols. For aerosol formulations one may use compressed gases suitable for this purpose, as are e.g. oxygen, nitrogen and carbon dioxide. The pharmaceutically useful agents may also contain additives for conservation, stabilisation, e.g. UV stabilizers, emulsifiers, sweetener, aromatisers, salts to change the osmotic pressure, buffers, coating additives and antioxidants.

A daily dosage per patient of about 1 mg to about 4000 mg especially about 50 mg to 3 g is usual with those of ordinary skill in the art appreciating that the dosage will depend also upon the age, conditions of the mammals, and the kind of diseases being treated or prevented. The daily dosage can be administrated in a single dose or can be divided over several doses. An average single dose of about 50 mg, 100 mg, 250 mg, 500 mg, 1000 mg and 2000 mg can be contemplated.

In the following the invention is described in more detail with reference to examples. These examples are intended for illustration only and are not to be construed as any limitation. The synthesis of the examples is described in WO03031443 and WO03032962.

### EXAMPLES

### Example 1

### 7-(4-{4-[5-(acetylamino-methyl)-2-oxo-oxazolidin-3-yl]-2-fluoro-phenyl}-piperazin-1-yl)-1-cyclopropyl-6-fluoro-4-oxo-1,4-dihydro-quinoline-3-carboxylic Acid

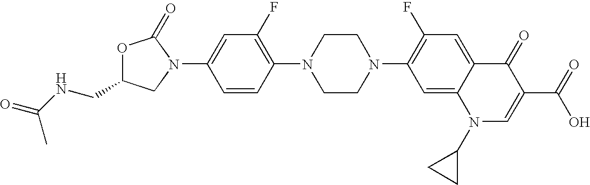

### Example 2

### 9-(4-{4-[5-(acetylamino-methyl)-2-oxo-oxazolidin-3-yl]-2-fluoro-phenyl}-piperazin-1-yl)-8-fluoro-3-methyl-6-oxo-2,3-dihydro-6H-1-oxa-3a-aza-phenalene-5-carboxylic Acid

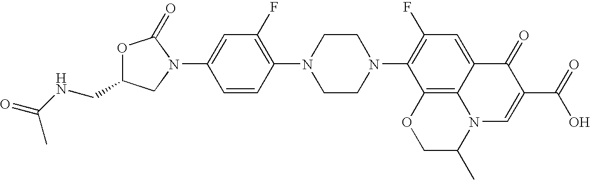

### Example 3

### 7-((3R,S)-3-{4-[(5S)-5-(acetylamino-methyl)-2-oxo-oxazolidin-3-yl]-2-fluoro-phenylcarbamoyl}-piperazin-1-yl)-1-cyclopropyl-6-fluoro-4-oxo-1,4-dihydro-quinoline-3-carboxylic acid.

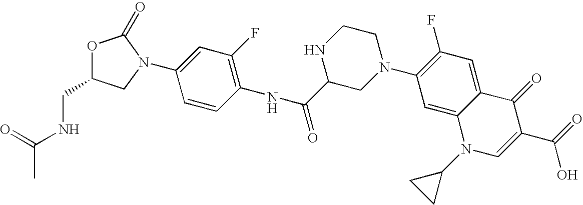

### Example 4

### 7-[(3R)-3-{4-((5S)-5-(Acetylamino-methyl)-2-oxo-oxazolidin-3-yl]-2-fluoro-phenylamino}-pyrrolidin-1-yl]-1cyclopropyl-6-fluoro-4-oxo-1,4-dihydro-quinoline-1-carboxylic Acid

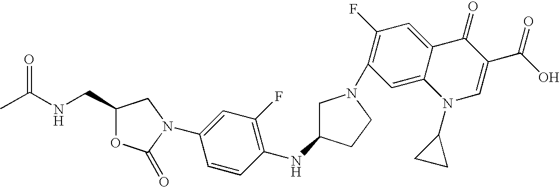

### Example 5

### 7-(4-{4-[(5S)-5-(Acetylamino-methyl)-2-oxo-oxazolidin-3-yl]-2-fluoro-phenyl}-piperazin-1-yl)-6-fluoro-1-(5-fluoro-pyridin-2-yl)-4-oxo-1,4-dihydro-quinoline-3-carboxylic Acid

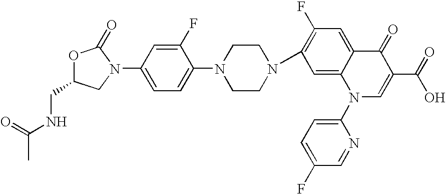

### Example 6

### 7-(4-{(5S)-5-(Acetylamino-methyl)-2-oxo-oxazolidin-3-yl]-2-fluoro-phenyl}-piperazin-1-yl)-1-(2,4-difluoro-phenyl)-6-fluoro-4-oxo-1,4-dihydro-quinoline-3-carboxylic Acid

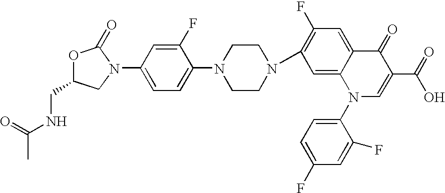

### Example 7

### 7-(4-{4-[(5S)-5-(Acetylamino-methyl)-2-oxo-oxazolidin-3-yl]-2-fluoro-phenyl}-piperazin-1-yl)-1-cyclopropyl-8-methoxy-4-oxo-1,4-dihydro-quinoline-3-carboxylic Acid

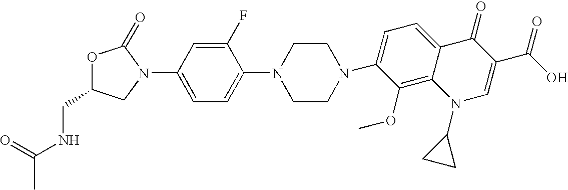

### Example 8

### 9-(4-{4-[(5S)-5-(Acetylamino-methyl)-2-oxo-oxazolidin-3-yl]-2-fluoro-phenyl}-piperazin-1-yl)-8-fluoro-3-methyl-6-oxo-2,3-dihydro-6H-1-oxa-3,3a-diaza-phenalene-5-carboxylic Acid

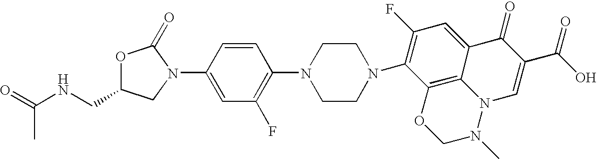

### Example 9

### 7-{(3RS)-3-[({4-[(5S)-5-(Acetylamino-methyl)-2-oxo-oxazolidin-3-yl]-2-fluoro-phenyl}-ethyl-amino)methyl]-piperazin-1-yl}-1-cyclopropyl-6-fluoro-4-oxo-1,4-dihydro-quinoline-3-carboxylic acid.

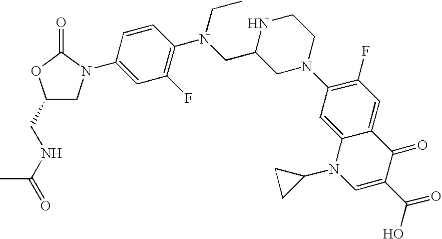

### Example 10

### 7-(4-{[(5S)-5-(Acetylamino-methyl)-2-oxo-oxazolidin-3-yl]-2-fluoro-phenyl}-piperazin-1-yl)-1-cyclopropyl-6-fluoro-4-oxo-1,4-dihydro-[1,8]naphthyridine-3-carboxylic Acid

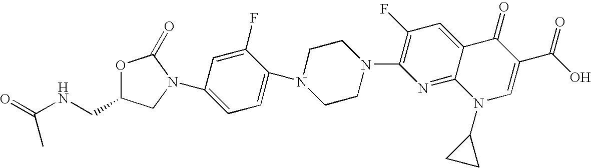

### Example 11

### 7-{4-[2-(4-{4-[5-(Acetylamino-methyl)-2-oxo-oxazolidin-3-yl]-2-fluoro-phenyl}-piperazin-1-yl)-ethyl]-piperazin-1-yl}-1-cyclopropyl-6-fluoro-4-oxo-1,4-dihydro-quinoline-3-carboxylic Acid

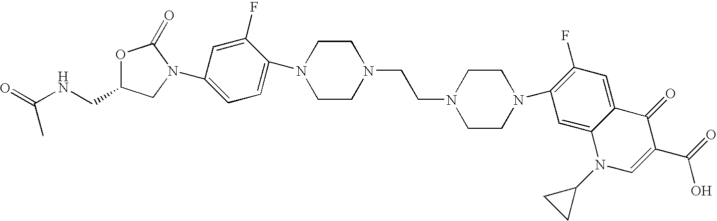

### Example 12

### 7-[4-(4-{4-[(5S)-5-(Acetylamino-methyl)-2-oxo-oxazolidin-3-yl]-2-fluoro-phenyl}-piperazin-1-yl)-piperidin-1-yl]-1-cyclopropyl-6-fluoro-4-oxo-1,4-dihydro-quinoline-3-carboxylic Acid

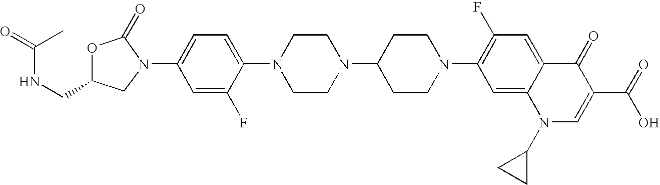

### Example 13

### 7-[(3R, 4R) and (3S, 4S)-3-{4-[(5S)-5-(Acetylamino-methyl)-2-oxo-oxazolidin-3-yl]-2-fluoro-phenyl}-4-aminomethyl-pyrrolidin-1-yl]-1-cyclopropyl-6-fluoro-4-oxo-1,4-dihydro-quinolin-3-carboxylic acid.

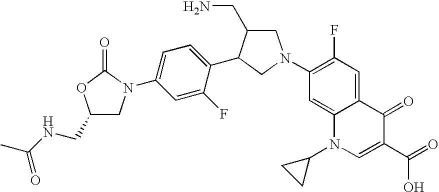

### Example 14

### 7-{4-[2-(4-{4-[(5S)-5-(Acetylamino-methyl)-2-oxo-oxazolidin-3-yl]-2-fluoro-phenyl}-piperazin-1-yl)-2-oxo-ethyl]-piperazin-1-yl}-1-cyclopropyl-6-fluoro-4-oxo-1,4-dihydro-quinolone-3-carboxylic Acid

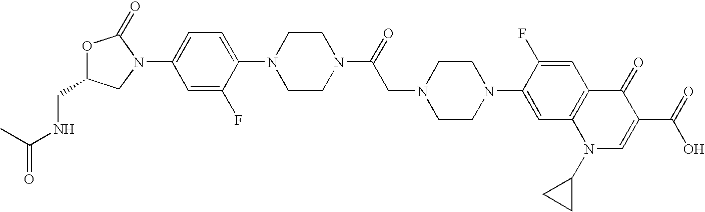

### Example 15

### 7-(3-{4-[5(S)-5-(Acetylamino-methyl)-2-oxo-oxazolidin-3-yl]-2-fluoro-phenylamino}-azetidin-1-yl)-1-cyclopropyl-6-fluoro-4-oxo-1,4-dihydro-[1,8]naphthyridine-3-carboxylic Acid

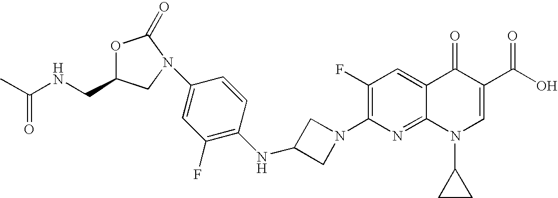

### Example 16

### 7-[(3R)-3-{4-[(5S)-5-(Acetylamino-methyl)-2-oxo-oxazolidin-3-yl]-2-fluoro-phenylamino}-pyrrolidin-1-yl]-1-cyclopropyl-6-fluoro-4-oxo-1,4-dihydro-[1,8]-naphthyridine-3-carboxylic Acid

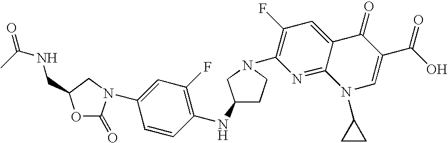

### Example 17

### 7-[(3R,4S) and (3S, 4R)-3-(−4{4-[(5S)-5-(Acetylamino-methyl)-2-oxo-oxazolidin-3-yl]-2-fluoro-phenyl}piperazine-1-carbonyl)-4-aminomethyl-pyrrolidin-1-yl]-1-cyclopropyl-6-fluoro-4-oxo-1,4-dihydro-quinoline carboxylic Acid

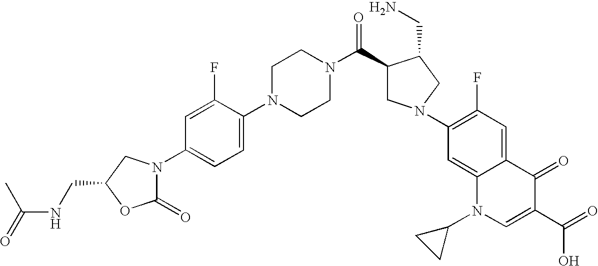

### Example 18

### 7-[(3R, 4S) and (3S, 4R)-3-(4-{4-[(5S)-5-(Acetylamino-methyl)-2-oxo-oxazolidin-3-yl]-2-fluoro-phenyl}-piperazine-1-carbonyl)-4-aminomethyl-pyrrolidin-1-yl)1-cyclopropyl-6-fluoro-4-oxo-1,4-dihydro-[1,8]naphthyridine-3-carboxylic Acid

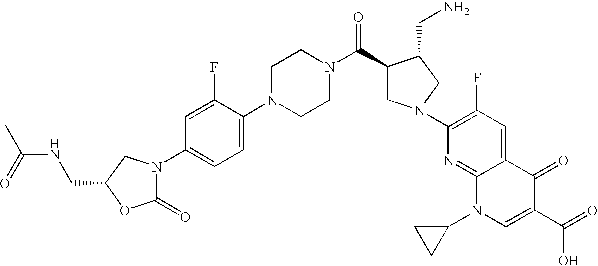

### Example 19

### 7-(4-{5-[(5S)-5-(Acetylamino-methyl)-2-oxo-oxazolidin-3-yl]-pyridin-2-yl}-1-piperazin-1-yl)-1-cyclopropyl-6-fluoro-4-oxo-1,4-dihydro-[1,8]naphthyridine-3-carboxylic Acid

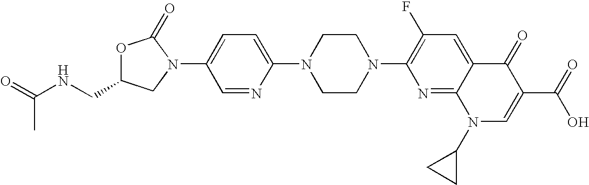

### Example 20

### 7-(4-{5-[(5S)-5-(Acetylamino-methyl)-2-oxo-oxazolidin-3-yl]-pyridin-2-yl}-piperazin-1-yl)-1-cyclopropyl-6-fluoro-4-oxo-1,4-dihydro-quinoline-3-carboxylic acid.

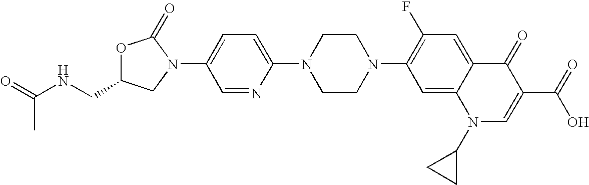

### Example 21

### 7-[(3R)-3-(4-{4[(5S)-5-(Acetylamino-methyl)-2-oxo-oxazolidin-3-yl]-2-fluoro-phenyl}-piperazin-1-yl)-pyrrolidin-1-yl]-1-cyclopropyl-6-fluoro-4-oxo-1,4-dihydro-[1,8]naphthyridine-3-carboxylic Acid

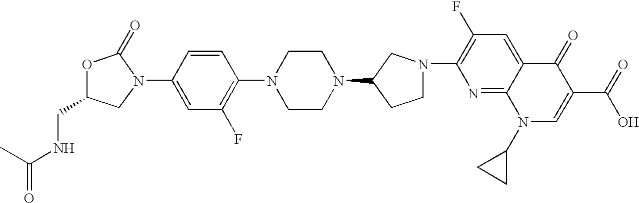

### Example 22

### 1-Cyclopropyl-6-fluoro-7-(4-{2-fluoro-4-[(5R)-5-(methansulfonylamino-methyl)-2-oxo-oxazolidin-3-yl]-phenyl}-piperazin-1-yl)-4-oxo-1,4-dihydro-[1,8]naphthyridine-3-carboxylic acid.

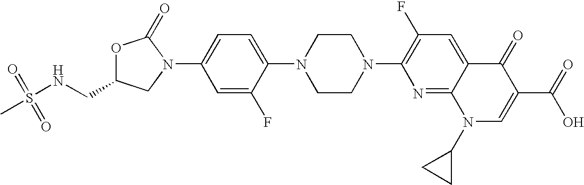

### Example 23

### 7-(4-{(4-[(5S)-5-(Acetylamino-methyl)-2-oxo-oxazolidin-3-yl]-2-fluoro-phenylamino}-piperidin-1-yl)-1-cyclopropyl-6-fluoro-4-oxo-1,4-dihydro-[1,8]naphthyridine-3-carboxylic Acid

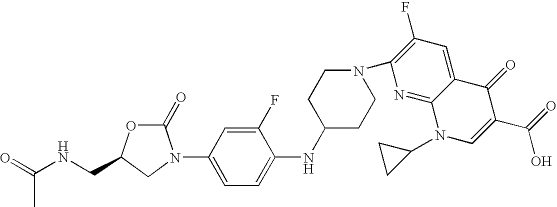

### Example 24

### 1-Cyclopropyl-6-fluoro-7-(4-[(2-fluoro-4-[(5S)-5-(methoxythiocarbonylamino-methyl)-2-oxo-oxazolidin-3-yl]-phenyl]-piperazin-1-yl)-4-oxo-1,4-dihydro-[1,8]-naphthyridine-3-carboxylic Acid

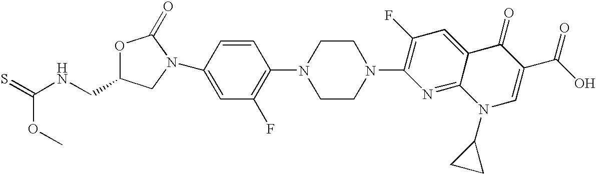

### Example 25

### 1-Cyclopropyl-6-fluoro-7-(4-{2-fluoro-4-((5S)-5-(methylsulfanylthiocarbonylamino-methyl)-2-oxo-oxazolidin-3-yl]-phenyl}-piperazin-1-yl)-4-oxo-1,4dihydro-[1,8]naphthyridine-3-carboxylic Acid

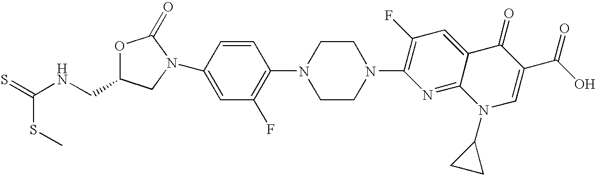

### Example 26

### 1-Cyclopropyl-6-fluoro-{4-[2-fluoro-4-{(5S)-2-oxo-5-thioureidomethyl-oxazolidin-3-yl}-phenyl]-piperazin-1-yl}-4-oxo-1,4-dihydro-[1,8]naphthyridine-3-carboxylic Acid

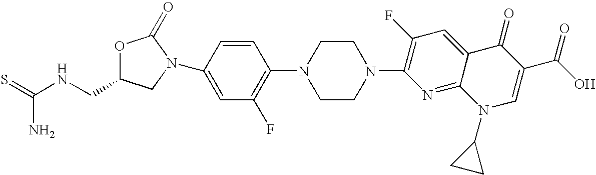

### Example 27

### 7-(4-{4-[5(S)-5-(Acetylamino-methyl)-2-oxo-oxazolidin-3-yl]-2-fluoro-phenoxy}-piperidin-1-yl)-1-cyclopropyl-6-fluoro-4-oxo-1,4-dihydro-[1,8]naphthyridine-3-carboxylic Acid

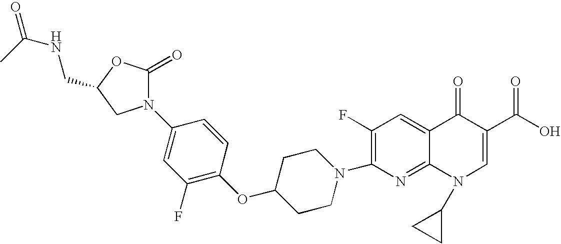

### Example 28

### 7-(4-{4-[5(S)-5-(Acetylamino-methyl)-2-oxo-oxazolidin-3-yl]-2-fluoro-phenoxy}-piperidin-1-yl)-1-cyclopropyl-6-fluoro-4-oxo-1,4-dihydro-quinoline-3-carboxylic Acid

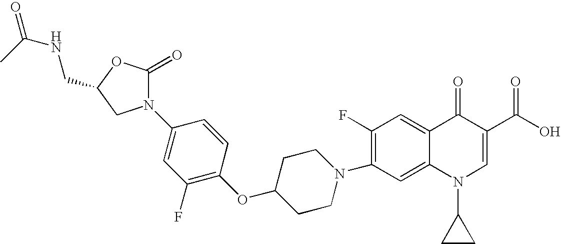

### Example 29

### 7-(4-{4-[5(S)-5-(Acetylamino-methyl)-2-oxo-oxazolidin-3-yl]-2-fluoro-phenylsulfinyl}-piperidin-1-yl)-1-cyclopropyl-6-fluoro-4-oxo-1,4-dihydro-quinoline-3-carboxylic Acid

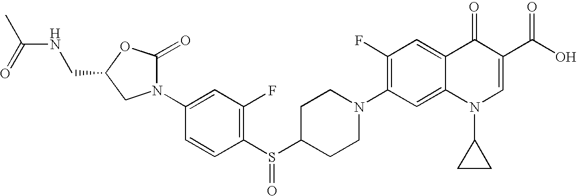

### Example 30

### 7-(4-{4-[5(S)-5(Acetylamino-methyl)-2-oxo-oxazolidin-3-yl]-2-fluoro-phenylsulfinyl}-piperidin-1-yl)-1-cyclopropyl-6-fluoro-4-oxo-1,4-dihydro-[1,8]naphthyridine-3-carboxylic Acid

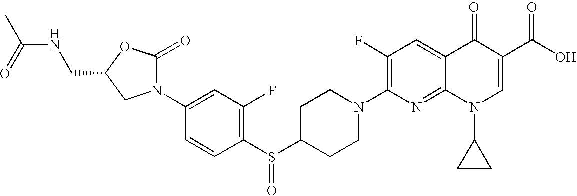

### Example 31

### 7-(3-{4-[5(S)-5-(Acetylamino-methyl)-2-oxo-oxazolidin-3-yl]-2-fluoro-phenoxy}-azetidin-1-yl)-1-cyclopropyl-6-fluoro-4-oxo-1,4-dihydro-[1,8]naphthyridine-3-carboxylic Acid

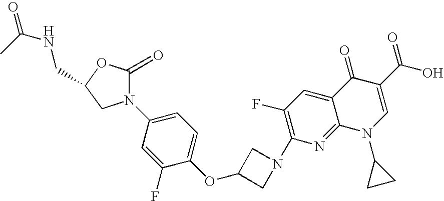

### Example 32

### 7-(3(S)-3-{4-[5(S)-5-(Acetylamino-methyl)-2-oxo-oxazolidin-3-yl]-2-fluoro-phenoxy}-pyrrolidin-1-yl)-1-cyclopropyl-6-fluoro-4-oxo-1,4-dihydro-[1,8]naphthyridine-3-carboxylic Acid

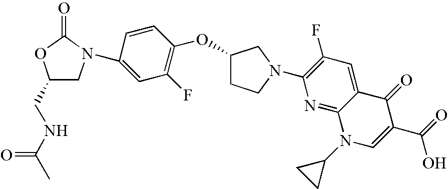

### Example 33

### 7-(3(S)-3-{4-[5(S)-5-(Acetylamino-methyl)-2-oxo-oxazolidin-3-yl]-2-fluoro-phenoxy}-pyrrolidin-1-yl)-1-cyclopropyl-6-fluoro-4-oxo-1,4-dihydro-quinoline-3-carboxylic Acid

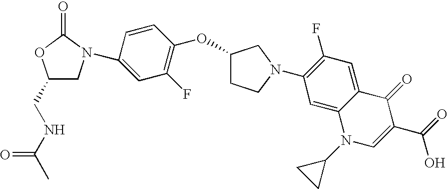

### Example 34

### 7-(4-{4-[5(S)-5-(Acetylamino-methyl)-2-oxo-oxazolidin-3-yl]-2-fluoro-phenoxymethyl}-piperidin-1-yl)-1-cyclopropyl-6-fluoro-4-oxo-1,4-dihydro-[1,8]naphthyridine-3-carboxylic Acid

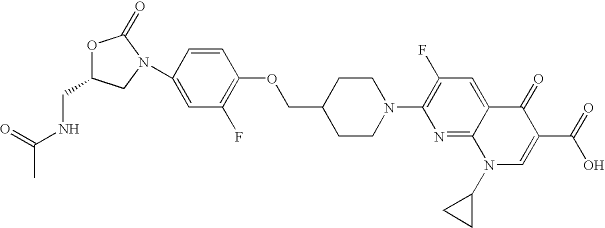

### Example 35

### 7-(4-{4-[5(S)-5-(Acetylamino-methyl)-2-oxo-oxazolidin-3-yl]-2-fluoro-phenoxymethyl}-piperidin-1-yl)-1-cyclopropyl-6-fluoro-4-oxo-1,4-dihydro-quinoline-3-carboxylic Acid

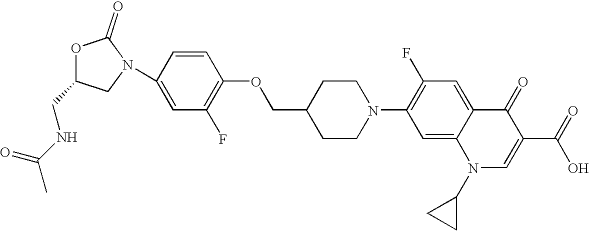

### Example 36

### 9-(3(S)-3-{4-[5(S)-5-(Acetylamino-methyl)-2-oxo-oxazolidin-3-yl]-2-fluoro-phenoxy}-pyrrolidin-1-yl)-8-fluoro-3-methyl-6-oxo-2,3-dihydro-6H-1-oxa-3a-aza-phenalene-5-carboxylic Acid

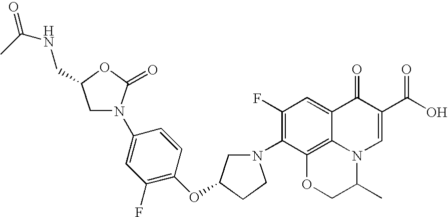

### Example 37

### 9-(4-{4-[5(S)-5-(Acetylamino-methyl)-2-oxo-oxazolidin-3-yl]-2-fluoro-phenoxy}-piperidin-1-yl)-8-fluoro-3-methyl-6-oxo-2,3-dihydro-6H-1-oxa-3a-aza-phenalene-5-carboxylic Acid

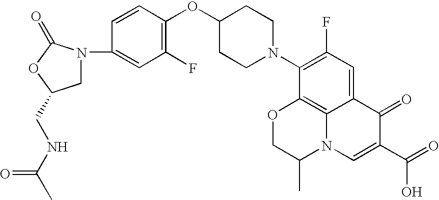

### Example 38

### 7-(3(R)-3-{4-[5(S)-5-(Acetylamino-methyl)-2-oxo-oxazolidin-3-yl]-2-fluoro-phenoxy}-pyrrolidin-1-yl)-1-cyclopropyl-6-fluoro-4-oxo-1,4-dihydro-[1,8]naphthyridine-3-carboxylic Acid

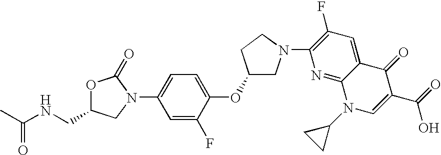

### Example 39

### 9-(3(R)-3-{4-[5(S)-5-(Acetylamino-methyl)-2-oxo-oxazolidin-3-yl]-2-fluoro-phenoxy}-azetidin-1-yl)-8-fluoro-3-methyl-6-oxo-2,3-dihydro-6H-1-oxa-3a-aza-phenalene-5-carboxylic Acid

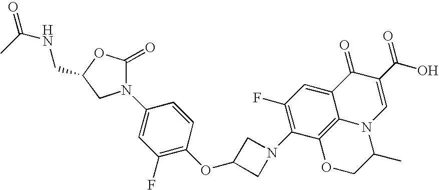

### Example 40

### 9-(4-{4-[5(S)-5-(Acetylamino-methyl)-2-oxo-oxazolidin-3-yl]-2-fluoro-phenylsulfanyl}-piperidin-1-yl)-8-fluoro-3-methyl-6-oxo-2,3-dihydro-6H-1-oxa-3a-aza-phenalene-5-carboxylic Acid

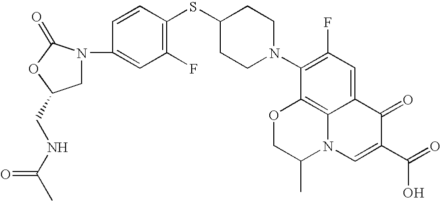

### Example 41

### 7-(4-{4-[5(S)-5-(Acetylamino-methyl)-2-oxo-oxazolidin-3-yl]-2-fluoro-phenylsulfanyl}-piperidin-1-yl)-1-cyclopropyl-6-fluoro-4-oxo-1,4-dihydro-[1,8]naphthyridine-3-carboxylic Acid

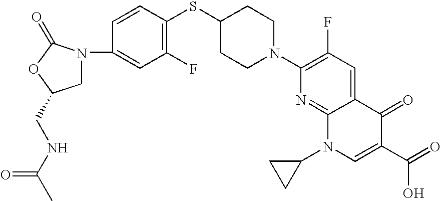

### Example 42

### 7-(3-{4-[5(S)-5-(Acetylamino-methyl)-2-oxo-oxazolidin-3-yl]-2-fluoro-phenoxymethyl}-pyrrolidin-1-yl)-1-cyclopropyl-6-fluoro-4-oxo-1,4-dihydro-[1,8]naphthyridine-3-carboxylic Acid

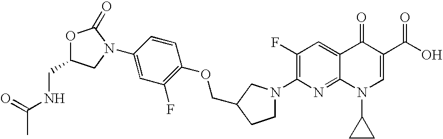

### Example 43

### 9-(3-{4-[5(S)-5-(Acetylamino-methyl)-2-oxo-oxazolidin-3-yl]-2-fluoro-phenoxymethyl}-pyrrolidin-1-yl)-8-fluoro-3-methyl-6-oxo-2,3-dihydro-6H-1-oxa-3a-aza-phenalene-5-carboxylic Acid

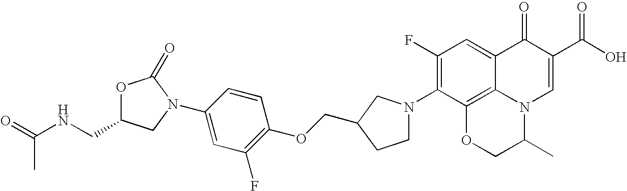

### Example 44

### 9-(4-{4-[5(S)-5-(Acetylamino-methyl)-2-oxo-oxazolidin-3-yl]-2-fluoro-phenoxymethyl}-piperidin-1-yl)-8-fluoro-3-methyl-6-oxo-2,3-dihydro-6H-1-oxa-3a-aza-phenalene-5-carboxylic Acid

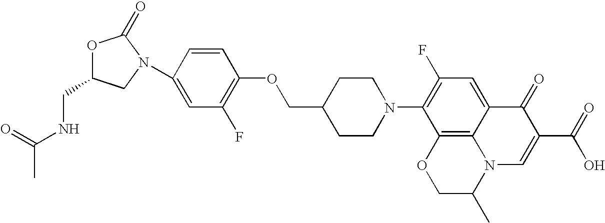

### Example 45

### 7-[4-(3-{4-[5(S)-5-(Acetylamino-methyl)-2-oxo-oxazolidin-3-yl]-2-fluoro-phenoxy}-propyl)-piperidin-1-yl]-1-cyclopropyl-6-fluoro-4-oxo-1,4-dihydro-[1,8]naphthyridine-3-carboxylic Acid

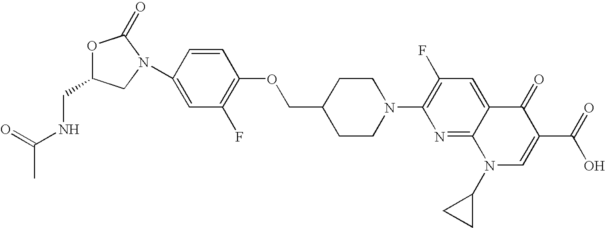

### Example 46

### 9-[4-(3-{4-[5(S)-5-(Acetylamino-methyl)-2-oxo-oxazolidin-3-yl]-2-fluoro-phenoxy}-propyl)-piperidin-1-yl]-8-fluoro-3-methyl-6-oxo-2,3-dihydro-6H-1-oxa-3a-aza-phenalene-5-carboxylic Acid

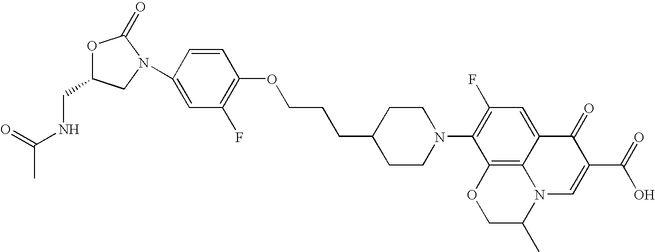

### Example 47

### 7-(4-{4-[5(S)-5-(Acetylamino-methyl)-2-oxo-oxazolidin-3-yl]-2-fluoro-phenoxy}-azepan-1-yl)-1-cyclopropyl-6-fluoro-4-oxo-1,4-dihydro-[1,8]naphthyridine-3-carboxylic Acid

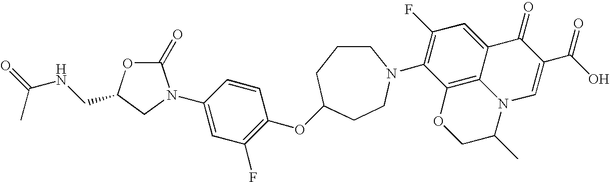

### Example 48

### 9-(4-{4-[5(S)-5-(Acetylamino-methyl)-2-oxo-oxazolidin-3-yl]-2-fluoro-phenoxy}-azepan-1-yl)-8-fluoro-3-methyl-6-oxo-2,3-dihydro-6H-1-oxa-3a-aza-phenalene-5-carboxylic Acid

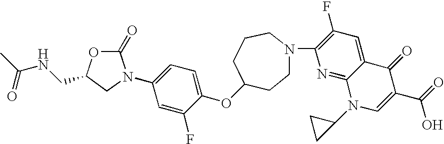

### Example 49

### 7-[4-(2-{4-[5(S)-5-(Acetylamino-methyl)-2-oxo-oxazolidin-3-yl]-2-fluoro-phenoxy}-ethyl)-piperidin-1-yl]-1-cyclopropyl-6-fluoro-4-oxo-1,4-dihydro-[1,8]naphthyridine-3-carboxylic Acid

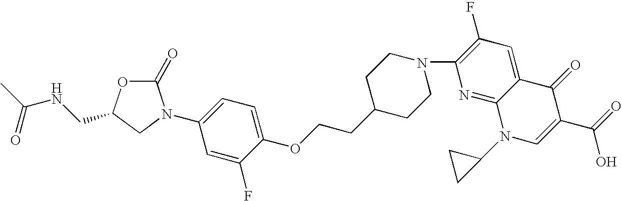

### Example 50

### 9-[4-(2-{4-[5(S)-5-(Acetylamino-methyl)-2-oxo-oxazolidin-3-yl]-2-fluoro-phenoxy}-ethyl)-piperidin-1-yl]-8-fluoro-3-methyl-6-oxo-2,3-dihydro-6H-1-oxa-3a-aza-phenalene-5-carboxylic Acid

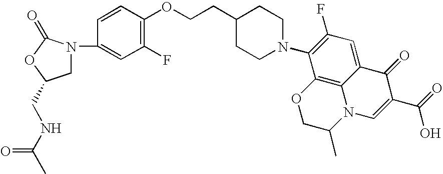

### Example 51

### 7-[3-(2-{4-[5(S)-5-(Acetylamino-methyl)-2-oxo-oxazolidin-3-yl]-2-fluoro-phenoxy}-ethyl)-pyrrolidin-1-yl]-1-cyclopropyl-6-fluoro-4-oxo-1,4-dihydro-[1,8]naphthyridine-3-carboxylic Acid

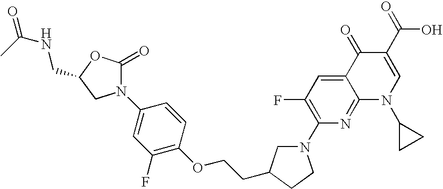

### Example 52

### 9-[3-(2-{4-[5(S)-5-(Acetylamino-methyl)-2-oxo-oxazolidin-3-yl]-2-fluoro-phenoxy}-ethyl)-pyrrolidin-1-yl]-8-fluoro-3-methyl-6-oxo-2,3-dihydro-6H-1-oxa-3a-aza-phenalene-5-carboxylic Acid

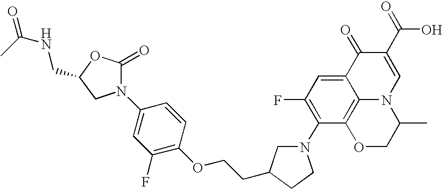

### Example 53

### 7-[3-(2-{4-[5(S)-5-(Acetylamino-methyl)-2-oxo-oxazolidin-3-yl]-2-fluoro-phenoxy}-ethyl)-pyrrolidin-1-yl]-1-cyclopropyl-6-fluoro-4-oxo-1,4-dihydro-quinoline-3-carboxylic Acid

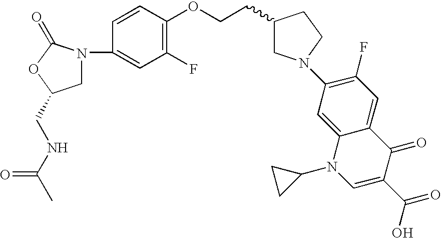

### Example 54

### 7-(3-{4-[5(S)-5-(Acetylamino-methyl)-2-oxo-oxazolidin-3-yl]-2-fluoro-phenoxymethyl}-pyrrolidin-1-yl)-1-cyclopropyl-8-methoxy-4-oxo-1,4-dihydro-quinoline-3-carboxylic Acid

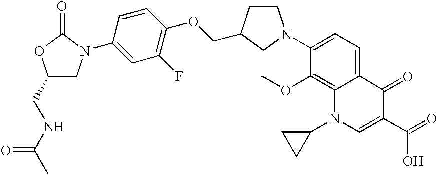

### Example 55

### 7-(3(S)-3-{4-[5(S)-5-(Acetylamino-methyl)-2-oxo-oxazolidin-3-yl]-2-fluoro-phenoxy)-pyrrolidin-1-yl)-6-fluoro-1-(4-hydroxy-phenyl)-4-oxo-1,4-dihydro-quinoline-3-carboxylic Acid

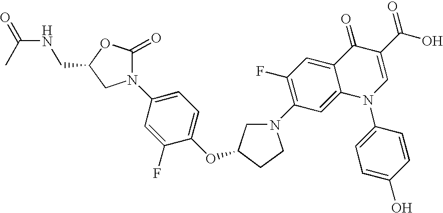

### Example 56

### 7-(3-{4-[5(S)-5-(Acetylamino-methyl)-2-oxo-oxazolidin-3-yl]-2-fluoro-phenoxymethyl}-pyrrolidin-1-yl)-1-cyclopropyl-6-fluoro-8-methoxy-4-oxo-1,4-dihydro-quinoline-3-carboxylic Acid

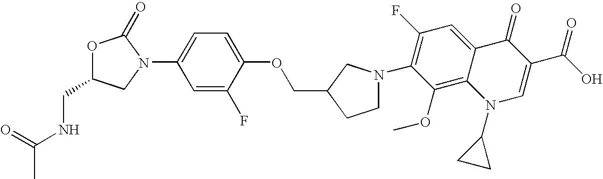

### Example 57

### 7-(3(S)-3-{4-[5(S)-5-(Acetylamino-methyl)-2-oxo-oxazolidin-3-yl]-2-fluoro-phenoxymethyl}-pyrrolidin-1-yl)-1-cyclopropyl-6-fluoro-4-oxo-1,4-dihydro-[1,8]naphthyridine-3-carboxylic Acid

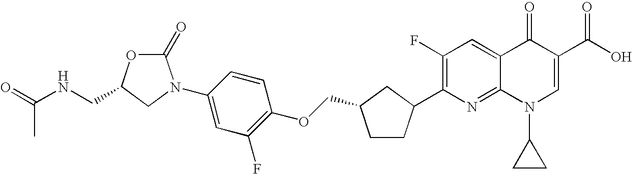

### Example 58

### 7-(3(R)-3-{4-[5(S)-5-(Acetylamino-methyl)-2-oxo-oxazolidin-3-yl]-2-fluoro-phenoxymethyl}-pyrrolidin-1-yl)-1-cyclopropyl-6-fluoro-4-oxo-1,4-dihydro-quinoline-3-carboxylic Acid

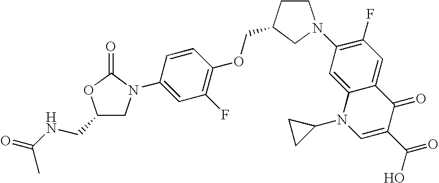

### Example 59

### 7-(3(R)-3-{4-[5(S)-5-(Acetylamino-methyl)-2-oxo-oxazolidin-3-yl]-2-fluoro-phenoxymethyl}-azetidin-1-yl)-1-cyclopropyl-6-fluoro-4-oxo-1,4-dihydro-[1,8]naphthyridine-3-carboxylic Acid

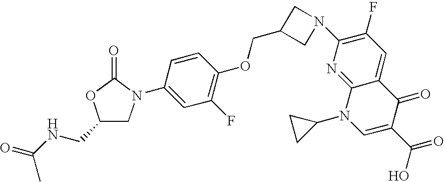

### Example 60

### 7-(3(R)-3-{4-[5(S)-5-(Acetylamino-methyl)-2-oxo-oxazolidin-3-yl]-2-fluoro-phenoxymethyl}-pyrrolidin-1-yl)-1-cyclopropyl-6-fluoro-4-oxo-1,4-dihydro-[1,8]naphthyridine-3-carboxylic Acid

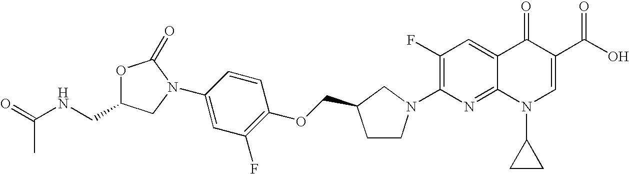

### Example 61

### 7-(3(R)-3-{4-[5(S)-5-(Acetylamino-methyl)-2-oxo-oxazolidin-3-yl]-2-fluoro-phenoxymethyl}-pyrrolidin-1-yl)-1-cyclopropyl-6-fluoro-4-oxo-1,4-dihydro-quinoline-3-carboxylic Acid

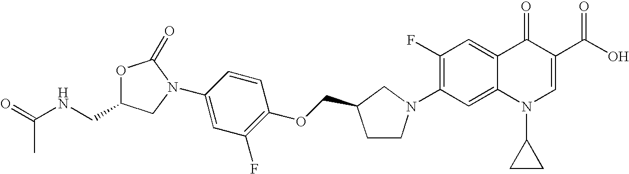

### Example 62

### 7-[4-(2-{4-[5(S)-5-(Acetylamino-methyl)-2-oxo-oxazolidin-3-yl]-2-fluoro-phenoxy}-ethyl)-piperidin-1-yl]-1-cyclopropyl-6-fluoro-4-oxo-1,4-dihydro-quinoline-3-carboxylic Acid

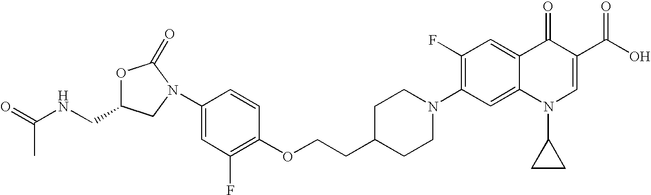

### Example 63

### 7-[3(S)-3-(2-{4-[5(S)-5-(Acetylamino-methyl)-2-oxo-oxazolidin-3-yl]-2-fluoro-phenoxy}-ethoxy)-pyrrolidin-1-yl]-1-cyclopropyl-6-fluoro-4-oxo-1,4-dihydro-quinoline-3-carboxylic Acid

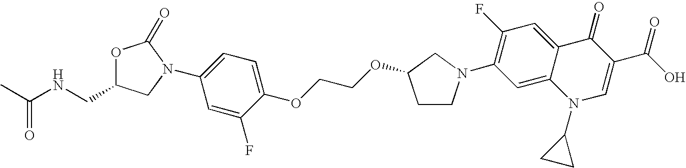

### Example 64

### 7-{3-[(2-{4-[5(S)-5-(Acetylamino-methyl)-2-oxo-oxazolidin-3-yl]-2-fluoro-phenoxy}-ethyl)-methyl-amino]-pyrrolidin-1-yl}-1-cyclopropyl-6-fluoro-4-oxo-1,4-dihydro-quinoline-3-carboxylic Acid

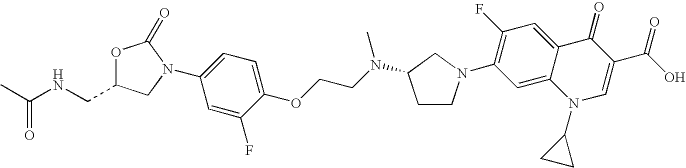

### Example 65

### 7-[3-(2-{4-[5(S)-5-(Acetylamino-methyl)-2-oxo-oxazolidin-3-yl]-2-fluoro-phenoxy}-ethylamino)-azetidin-1-yl]-1-cyclopropyl-6-fluoro-4-oxo-1,4-dihydro-[1,8]naphthyridine-3-carboxylic Acid

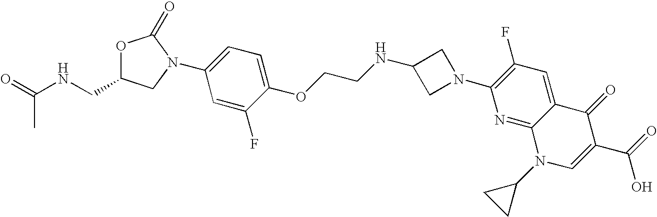

### Example 66

### 7-[3-(2-{4-[5(S)-5-(Acetylamino-methyl)-2-oxo-oxazolidin-3-yl]-2-fluoro-phenoxy}-ethylamino)-azetidin-1-yl]-1-cyclopropyl-6-fluoro-4-oxo-1,4-dihydro-quinoline-3-carboxylic Acid

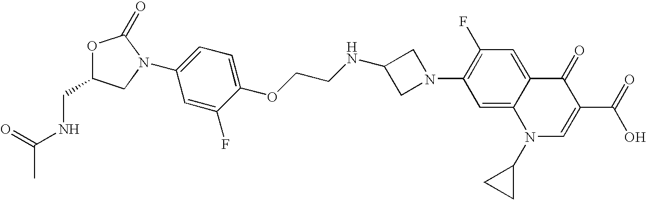

### Example 67

### 7-[1-(2-{4-[5(S)-5-(Acetylamino-methyl)-2-oxo-oxazolidin-3-yl]-2-fluoro-phenoxy}-ethyl)-octahydro-pyrrolo[2,3-c]pyridin-6-yl]-1-cyclopropyl-6-fluoro-8-methoxy-4-oxo-1,4-dihydro-quinoline-3-carboxylic Acid

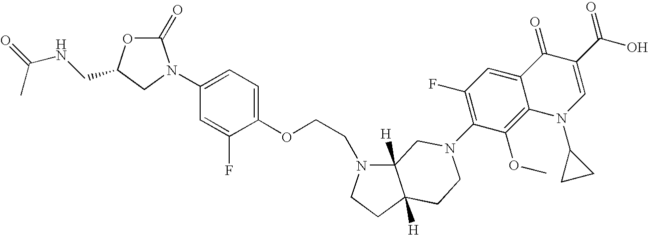

### Example 68

### 7-(3(S)-3-{4-[5(S)-5-(Acetylamino-methyl)-2-oxo-oxazolidin-3-yl]-2-fluoro-phenoxymethyl}-pyrrolidin-1-yl)-1-(2,4-difluoro-phenyl)-6-fluoro-4-oxo-1,4-dihydro-quinoline-3-carboxylic Acid

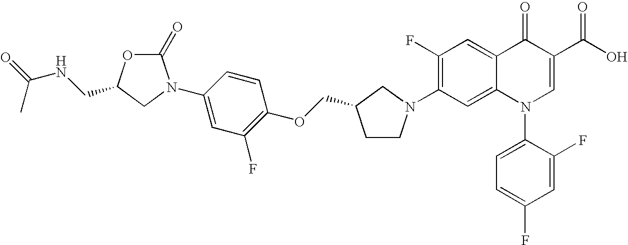

### Example 69

### 7-(3(R)-3-{4-[5(S)-5-(Acetylamino-methyl)-2-oxo-oxazolidin-3-yl]-2-fluoro-phenoxymethyl}-pyrrolidin-1-yl)-6-fluoro-1-(5-fluoro-pyridin-2-yl)-4-oxo-1,4-dihydro-quinoline-3-carboxylic Acid

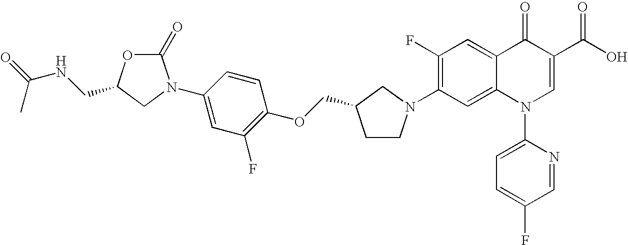

### Example 70

### 7-(3-{4-[5(S)-5-(Acetylamino-methyl)-2-oxo-oxazolidin-3-yl]-2-fluoro-phenoxymethyl}-pyrrolidin-1-yl)-1-cyclopropyl-8-methoxy-4-oxo-1,4-dihydro-quinoline-3-carboxylic Acid

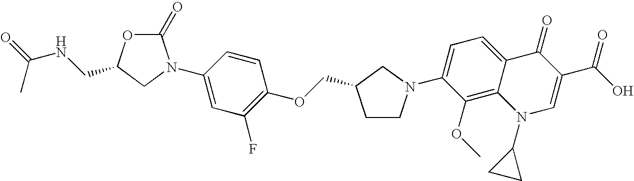

### Example 71

### 7-(3-{4-[5(S)-5-(Acetylamino-methyl)-2-oxo-oxazolidin-3-yl]-2-fluoro-phenoxymethyl)-pyrrolidin-1-yl)-1-cyclopropyl-6-fluoro-8-methoxy-4-oxo-1,4-dihydro-quinoline-3-carboxylic Acid

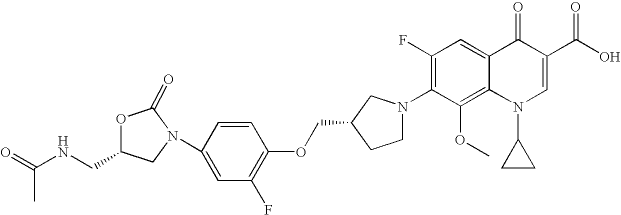

### Example 72

### 9-(3-{4-[5(S)-5-(Acetylamino-methyl)-2-oxo-oxazolidin-3-yl]-2-fluoro-phenoxymethyl}-pyrrolidin-1-yl)-8-fluoro-3-methyl-6-oxo-2,3-dihydro-6H-1-oxa-3a-aza-phenalene-5-carboxylic Acid

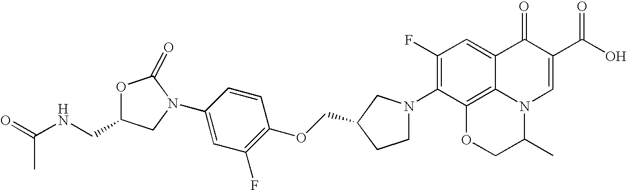

### Example 73

### 7-(3-{4-[5(S)-5-(Acetylamino-methyl)-2-oxo-oxazolidin-3-yl]-2-fluoro-phenoxymethyl}-pyrrolidin-1-yl)-1-(2,4-difluoro-phenyl)-6-fluoro-4-oxo-1,4-dihydro-quinoline-3-carboxylic Acid

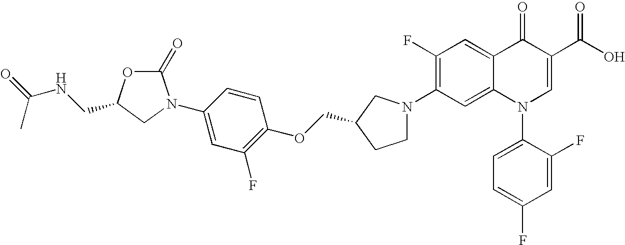

### Example 74

### 7-(4-{4-[5(S)-5-(Acetylamino-methyl)-2-oxo-oxazolidin-3-yl]-2-fluoro-phenoxymethyl}-piperidin-1-yl)-1-cyclopropyl-6-fluoro-8-methoxy-4-oxo-1,4-dihydro-quinoline-3-carboxylic Acid

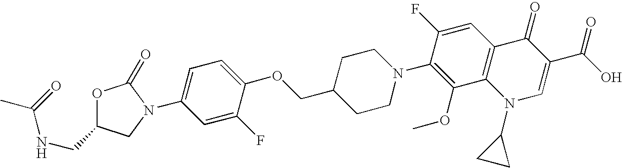

### Example 75

### 7-(4-{4-[5(S)-5-(Acetylamino-methyl)-2-oxo-oxazolidin-3-yl]-2-fluoro-phenoxy}-azepan-1-yl)-1-cyclopropyl-6-fluoro-4-oxo-1,4-dihydro-quinoline-3-carboxylic Acid

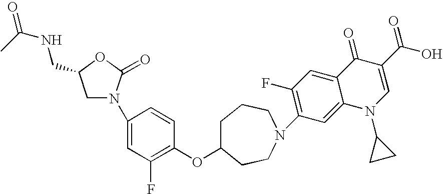

### Example 76

### 7-(3-{4-[5(S)-5-(Acetylamino-methyl)-2-oxo-oxazolidin-3-yl]-2-fluoro-phenoxymethyl}-pyrrolidin-1-yl)-1-(6-amino-3,5-difluoro-pyridin-2-yl)-8-chloro-6-fluoro-4-oxo-1,4-dihydro-quinoline-3-carboxylic Acid

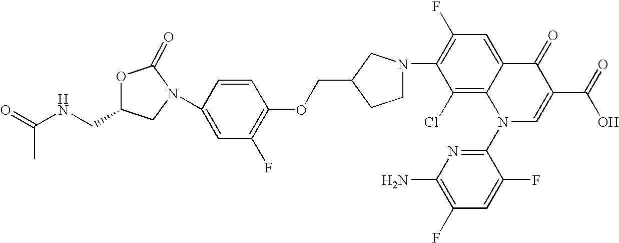

### Example 77

### 7-(4-{4-[5(S)-5-(Acetylamino-methyl)-2-oxo-oxazolidin-3-yl]-2-fluoro-phenoxymethyl}-azepan-1-yl)-1-cyclopropyl-6-fluoro-4-oxo-1,4-dihydro-[1,8]naphthyridine-3-carboxylic Acid

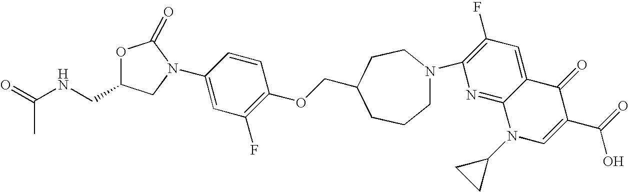

### Example 78

### 7-(4-{4-[5(S)-5-(Acetylamino-methyl)-2-oxo-oxazolidin-3-yl]-2-fluoro-phenoxymethyl}-azepan-1-yl)-1-cyclopropyl-6-fluoro-4-oxo-1,4-dihydro-quinoline-3-carboxylic Acid

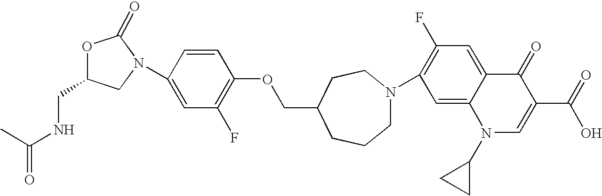

Microbiogical Methods

MICs (minimal inhibitory concentrations) were determined by a microdilution method following NCCLS guidelines, except that IsoSensitest broth (Oxoid; Basingstroke, UK) was used (National Committee for Clinical Laboratory Standards “Methods for Dilution Antimicrobial Susceptibility Tests for Bacteria that Grow Aerobically”, 4^{th} ed.; Approved standard: NCCLS Document M7-A4; National Committee for Clinical Laboratory Satandars: Villanova, Pa., USA, 1997). The medium was supplemented with 3% lysed horse blood for fastidious organisms. Serial dilutions in 96-well plates were prepared with the help of a Biomek 2000 robot. The pH of the test medium was varied from 7.8 to 6.4. The bacterial strains used were from the Morphochem collection. Clinical isolates were originally obtained from the Kantonspital Basel, Switzerland, and from other European and US hospitals.

pH-dependent antibacterial activity (MIC (μg/mL) at pH):

Example

| | 6.4 | 6.8 | 7.4 | 7.8 |
|---|---|---|---|---|
| linezolid | 0.5 | 0.5 | 1 | 1 |
| ciprofloxacin | 32 | 32 | 16 | 16 |
| 1 | 0.03 | 0.125 | 0.25 | 0.5 |
| 4 | 0.03 | 0.06 | 0.25 | 0.5 |
| 10 | 0.125 | 0.25 | 0.5 | 1 |

1. Use of a compound of Formula (I): 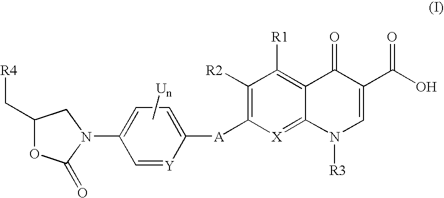
wherein
A is a bond, a NH, O, S, SO, SO₂, SO₂NH, PO₄, —NH—CO—NH—, —CO—NH—, —CO—, —CO—O—, —NH—CO—O—, an alkylene group, an alkenylene group, an alkinylene group, a heteroalkylene group, an arylene group, a heteroarylene group, a cycloalkylene group, a heterocycloalkylene group, an alkylarylene group or a heteroarylalkylene group or a combination of two or more of these atoms or groups;
X is CR5 or N;
Y is CR6 or N;
U is F or Cl;
n is 0, 1, 2 or 3;
R1 is H, F, Cl, Br, I, OH, NH₂, an alkyl group or a heteroalkyl group;
R2 is H, F or Cl;
R3 is H, an alkyl group, an alkenyl group, an alkinyl group, a heteroalkyl group, a cycloalkyl group, a heterocycloalkyl group, an aryl group, a heteroaryl group, an alkylaryl group or a heteroarylalkyl group;
R4 is a heteroalkyl group, a cycloalkyl group, a heterocycloalkyl group, an aryl group, a heteroaryl group, an alkylaryl group or a heteroarylalkyl group;
R5 is H, F, Cl, OH, NH₂, an alkyl group or a heteroalkyl group, or R3 and R5 can be linked via an alkylene, an alkenylene or a heteroalkylene group or be a part of a cycloalkylene or heterocycloalkylene group, in case R3 is no H and R5 is no H, F, OH, NH₂ or Cl;
R6 is H, F, Cl or OMe;
or a pharmacologically acceptable salt, solvate, hydrate or formulation thereof for the treatment of bacterial infections at a pH lower than 7.4.

2. Use of a compound according to claim 1, wherein R1 is H.

3. Use of a compound according to claim 1, wherein R2 is F.

4. Use of a compound according to claim 1, wherein R3 is an optionally substituted cyclopropyl group.

5. Use of a compound according to claim 1, wherein R4 is an optionally substituted acetylamino group.

6. Use of a compound according to claim 1, wherein the absolute configuration at C-5 of the oxazolidinone ring is (S) according to the Cahn-Ingold-Prelog nomenclature system.

7. Use of a compound according to claim 1, wherein X is N or CH.

8. Use of a compound according to claim 1, wherein Y is CF.

9. Use of a compound according to claim 1, wherein n is 0.

10. Use of a compound according to claim 1, wherein A is a group of the formula
—B₀₋₁[D-E₀₋₁]ₘ-G₀₋₁-K₀₋₁—
wherein
the group B is NH, O, S, SO, SO₂, SO₂NH, an alkylene, which may be substituted by one, two or more fluorine atoms, or a heteroalkylene group, which may be substituted by one, two or more fluorine atoms and/or at the optionally present nitrogen atoms by an alkyl or an acyl group;
the groups D independently of each other are optionally anellated heterocycloalkylen groups with 1, 2, 3 or 4 nitrogen atoms, which heterocycloalkylen groups may each be substituted by one, two or more fluorine atoms and/or which each may be substituted at one, two, three or four nitrogen atoms by an alkyl or an acyl group;
the groups E independently of each other are an alkylene, which may be substituted by one, two or more fluorine atoms, an O, S, SO, SO₂, SO₂NH group, or a heteroalkylen group, which may be substituted by one, two or more fluorine atoms and/or at the optionally present nitrogen atoms by an alkyl or an acyl group;
the group G contains one or more optionally anellated heterocycloalkylene groups with 1, 2, 3 or 4 nitrogen atoms, which heterocycloalkylene groups may each be substituted by one, two or more fluorine atoms and/or which each may be substituted at one, two, three or four nitrogen atoms by an alkyl or an acyl group;
the group K is an alkylene, which may be substituted by one, two or more fluorine atoms, an O, S, SO, SO₂, SO₂NH group, or a heteroalkylene group, which may be substituted by one, two or more fluorine atoms and/or at the optionally present nitrogen atoms by an alkyl or an acyl group; and m=1, 2, 3 or 4.

11. Use of a compound according to claim 1, wherein A is a group of the formula —V—W—, wherein V is a direct bond or a group of the formula NH, O, S, SO, SO₂, SO₂NH, PO₄, —NH—CO—NH—, —CO—NH—, —CO—, —CH₂—, —CO—O—, —(CH₂)₁₋₃—O—, —CH═CH—C(O)—, or —NH—CO—O— and W is a heterocycloalkyl group with 4 to 7 ring atoms or a alkylheterocycloalkyl group with 4 to 7 ring atoms and 1 to 4 carbon atoms in the alkyl chain, all these groups may be substituted by 1, 2, 3 or 4 fluorine atoms, methyl or methoxy groups.

12. Use of a compound according to claim 1, wherein A is a group of the formula 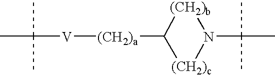
wherein
V is a group of the formula NH, O, S, SO, SO₂, SO₂NH, PO₄, —NH—CO—NH—, —CO—NH—, —CO—, —CH₂—, —CO—O—, —(CH₂)₁₋₃—O—, —CH═CH—C(O)—, or —NH—CO—O—; a is 0, 1, 2, 3 or 4; b is 0, 1, 2, 3 or 4; c is 0, 1, 2, 3 or 4 and 1, 2, 3 or 4 hydrogen atoms may be substituted by F, a methyl- or a methoxy group.

13. Use of a compound according to claim 12, wherein V is NH, O, S, SO or SO₂.

14. Use of a compound according to claim 12, wherein V is O or NH; a is 0 or 1; b is 1 or 2 and c is 1 or 2.

15. Use of a compound according to claim 1, wherein A is selected from the following groups which may be substituted by one, two or more fluorine atoms or by an alkyl group which may be substituted by one or more fluorine atoms, and wherein the amino groups may be substituted by an alkyl or an acyl group:

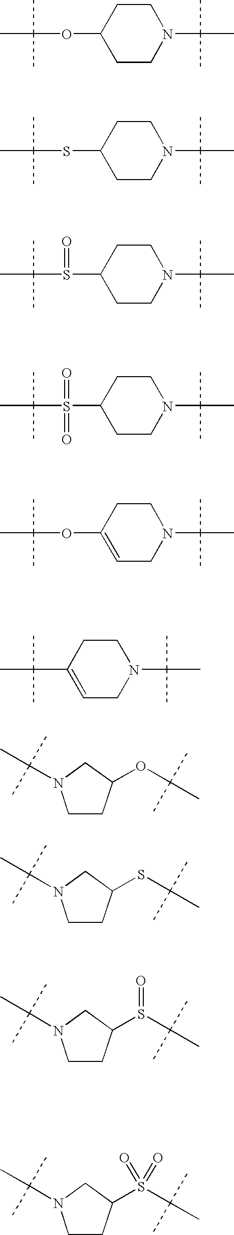

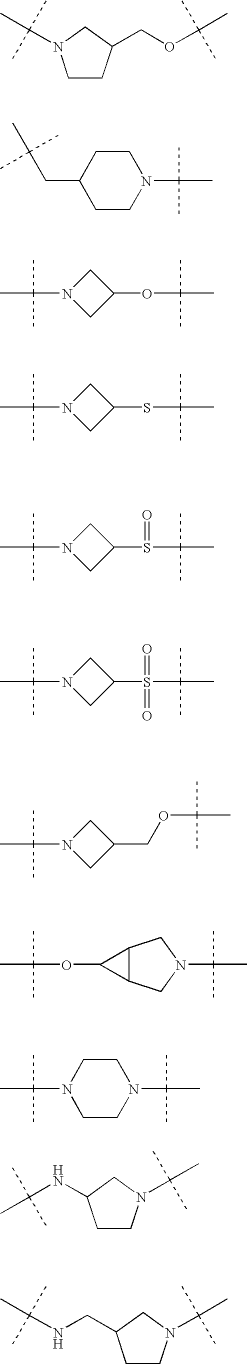

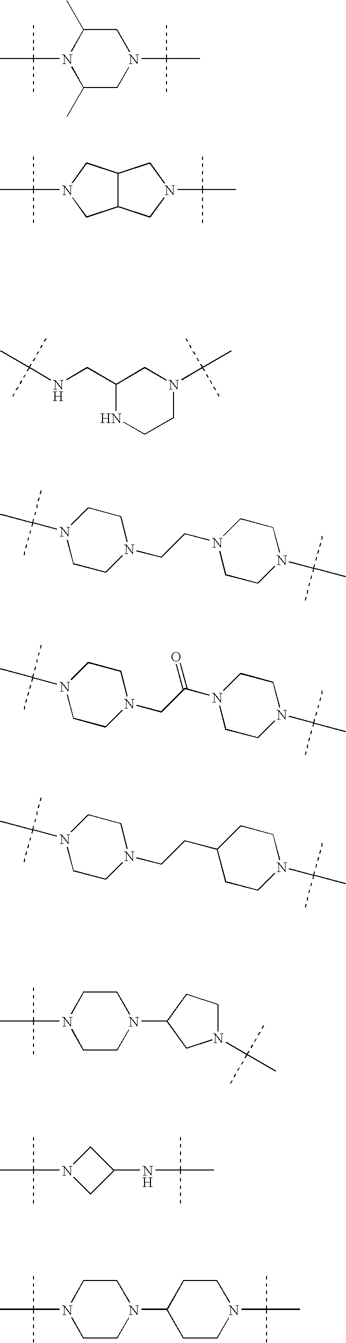

16. A pharmaceutical composition containing a compound according to claim 1 and optionally carriers and/or adjuvants and/or diluents.

17. Pro-drugs which contain a compound according to claim 1 and at least one pharmacologically acceptable protective group.

18. Use of a compound, a pharmaceutical composition or a pro-drug according to claim 1 for the manufacture of medicaments for the treatment of bacterial infections in environments having a pH<7.4, preferably <7.0.

19. Use of a compound, a pharmaceutical composition or a pro-drug according to claim 1 for the manufacture of medicaments for the treatment of bacterial infections in inflamed tissues and/or abscesses.
